(19) United States
(12) Patent Application Publication
(10) Pub. No.: US 2021/0020312 A1
(43) Pub. Date: Jan. 21, 2021

(54) EFFICIENT AND LIGHTWEIGHT PATIENT-MORTALITY-PREDICTION SYSTEM WITH MODELING AND REPORTING AT TIME OF ADMISSION

(71) Applicant: Regents of the University of Minnesota, Minneapolis, MN (US)

(72) Inventor: Nishant Sahni, St. Paul, MN (US)

(21) Appl. No.: 16/932,368

(22) Filed: Jul. 17, 2020

Related U.S. Application Data
(60) Provisional application No. 62/875,341, filed on Jul. 17, 2019.

(51) Int. Cl. G16H 50/20; G16H 10/60; G16H 50/30; G16H 50/50; G16H 10/40; G16H 15/00; A61B 5/00; A61B 5/145

(52) U.S. Cl. CPC G16H 50/20; G16H 10/60; G16H 50/30; G16H 50/50; G16H 10/40; A61B 5/0022; A61B 5/7275; A61B 5/4842; A61B 5/7264; A61B 5/14546; G16H 15/00

(57) ABSTRACT

This disclosure describes a “lightweight” model configured to accurately predict a patient's death within six months of hospital admission using only a limited number of data inputs. In some examples, a computing system executes a mortality prediction engine configured to apply a machine-learning model trained to predict mortality of the patient at a time period after admission of the patient, wherein the machine-learning model is trained based on training data that configures the mortality prediction engine to predict the mortality of the patient using a set of data parameters consisting of only: (1) data parameters available at admission of the patient, (2) a data parameter indicative of presence of a metastatic disease in the patient and (3) a data parameter indicative of presence of at least one active tumor in the patient.

This application claims the benefit of U.S. Provisional Patent Application No. 62/875,341, filed Jul. 17, 2019, the entire content being incorporated herein by reference.

### TECHNICAL FIELD

The disclosure relates to patient-mortality prediction systems for hospitals.

### BACKGROUND

Predicting death in a cohort of clinically diverse, multi-condition, hospitalized patients is difficult. This complexity frequently hinders timely serious-illness care conversations. Prognostic models that can determine, as one example, a six-month death risk at the time of hospital admission can improve access to serious-illness care conversations.

### SUMMARY

This disclosure describes a mortality prediction system that implements unique models to predict in-hospital mortality using easily available metrics at the time of admission to facilitate care. More specifically, example mortality-prediction systems are described that apply a unique random-forest model, referred to generally herein as a “mini Serious-Illness Algorithm” or “minSIA,” which, in one example, uses eight variables that may be easily obtained from data determined within the initial 48 hours of hospitalization to accurately predict the risk of a patient's death within six months. In this way, the system can identify patients at higher risk of six-month mortality at the time of hospital admission. This can be used to improve access to timely serious-illness care conversations in high-risk patients.

In general, there is a lack of accurate prognostic models that, at the time of hospital admission of multi-condition patients, predict patient mortality over a significant span of time after admission, such as a six-month mortality risk. This disclosure describes the development and validation of a user-friendly, lean, machine-learning model (e.g., minSIA) that accurately risk-stratifies patients at the time of hospital admission. Experimental results indicate that minSIA exhibits a remarkable Area Under the Curve (AUC) value of 0.92 for predicting patient death within the six-month period after hospital admission. This ability exceeds other AUCs of about 0.60-0.85 as previously reported for clinician performance in the literature of this field. The minSIA model is an innovative advancement that can potentially facilitate timely serious-illness care conversations in appropriate situations.

As shown herein, the system utilizes demographic data, vital-sign data, and laboratory data from the first 48 hours of hospitalization to accurately quantify a six-month mortality risk for a newly admitted patient. The minSIA algorithms described herein can identify patients at high risk of six-month mortality at the time of hospital admission. The systems and techniques of this disclosure can be used to facilitate timely serious-illness care conversations with high-risk patients.

### BRIEF DESCRIPTION OF DRAWINGS

FIG. 1 is a block diagram illustrating an example computing system for predicting in-hospital mortality for patients, in accordance with techniques of the present disclosure.

FIG. 2 is a bar graph illustrating example feature importances, determined and used as described herein, in the random-forest models applied by the computing system of FIG. 1.

FIG. 3A is a graph illustrating the Receiver Operating Characteristic (ROC) curve for the minSIA model described herein.

FIG. 3B is a graph illustrating the observed rate of death at one year from hospitalization for each of the 10 probability bins plotted on the x-axis. The predicted probability from the random-forest model is indicated on the y-axis. The dotted diagonal line represents points along a perfectly calibrated model. Each point on the graph represents one of the 10 bins of probability. The bars delineate the 95% confidence intervals around the observed probability.

FIG. 4A is a graph illustrating a “recall plot” indicating the percentage of the overall number of cases in a given category “gained” (y-axis) when applying the minSIA8 and selecting the highest k-deciles (x-axis). For example, if the positivity threshold is set to be the highest-ranking 20% cases (by predicted probability) then 83% percent of true-positives would be selected.

FIG. 4B is a graph illustrating an “accuracy plot” indicating the accuracy (y-axis) of the model at each decile threshold of predicted probability (x-axis).

FIG. 5 is a block diagram illustrating an example of various devices that may be configured to implement one or more techniques of the present disclosure.

### DETAILED DESCRIPTION

This disclosure describes a mortality-prediction system that implements unique models to predict in-hospital mortality using easily available metrics to facilitate and coordinate care (e.g., triage patients appropriately). More specifically, example mortality-prediction systems are described that apply a unique random-forest model, referred to herein as a “mini Serious Illness Algorithm,” which, in one example, uses eight variables easily obtained from data collected within the initial 48 hours of hospitalization in order to accurately predict a risk of patient death within six months from hospitalization. In this way, the system can identify, at the time of hospital admission, patients at higher risk of six-month mortality. The systems and techniques herein can be used to facilitate timely serious-illness care conversations in high-risk patients.

Accurate and timely prognostication is essential for ensuring that seriously ill patients receive care that is concordant with their goals and values—a critical component of high-quality care. Early conversations about advance care planning (ACP) with seriously ill patients can improve outcomes for patients and caregivers alike. However, serious-illness care conversations often occur too late, such as when patients are in crisis or unable to make decisions for themselves. Of those patients admitted to hospitals, fewer than half who need palliative care actually receive it. One of the major barriers to timely serious-illness care conversations is the relatively poor prognostic performance of clinicians in predicting longer-term mortality. This may be especially true for patients who have a number of serious, chronic medical conditions. Such patients constitute the largest proportion of hospice utilizers in the US. Timely prognostication and referral remains the Achilles heel for timely serious-illness discussions.

Despite prior efforts, a relatively simple predictive model to accurately prognosticate six-month mortality in diverse, multi-condition patients at the time of hospital admission has remained elusive. Existing prognostic models in this area frequently rely extensively on variables that may not be available to clinicians at the time of hospital admission, and/or use a very large number of variables, making them unwieldy to use. The techniques of this disclosure describe a user-friendly predictive model that estimates the probability of, as one example, six-month patient mortality at the time of hospital admission. Such a model will facilitate the objective and timely identification of high-risk, multi-condition, hospitalized patients.

A retrospective study was designed using electronic medical record data linked with the state death registry, including 158,323 hospitalized patients within a six-hospital network over a six-year period. Primary factors included the first set of vital signs, complete blood count, basic and complete metabolic panels, serum lactate, pro-BNP, troponin-I, INR, activated partial thromboplastin time (aPTT), demographic information, and associated ICD codes. The outcome of interest was patient death within six months. Model performance was measured on the validation dataset. A random-forest model, referred to herein as the “mini Serious-Illness Algorithm” or “minSIA,” used 8 variables from the initial 48 hours of hospitalization and predicted death within six months with an Area Under the Curve (AUC) value of about 0.92 (e.g., from 0.91 to 0.93). Red cell distribution width was one prognostic variable. In example experiments, minSIA was very well calibrated and estimated the probability of death to within 10% of the actual value. The discriminative ability of minSIA was significantly better than historical estimates of clinician performance.

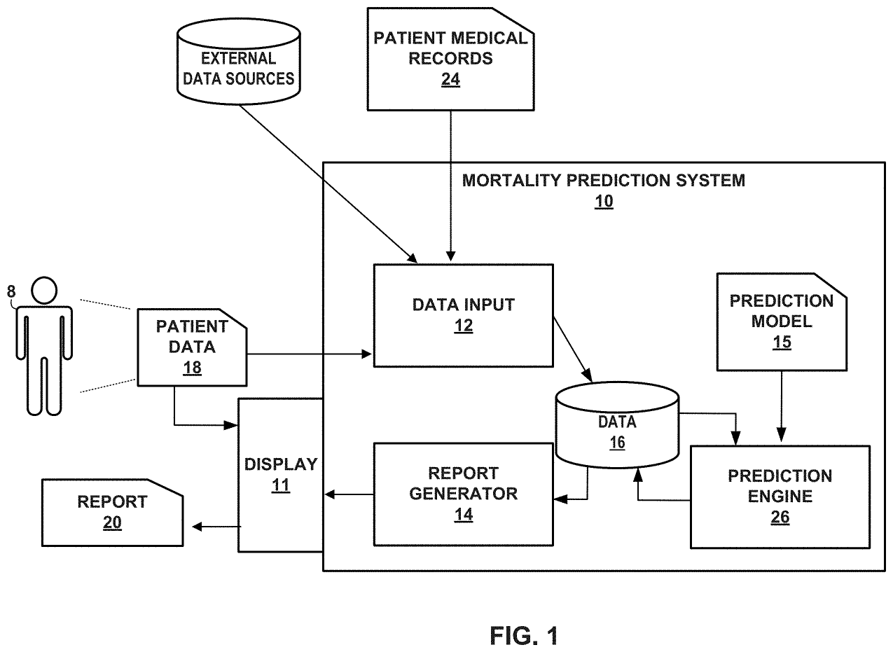

FIG. 1 is a block diagram illustrating an example computing system 10 for predicting in-hospital mortality for patients, in accordance with one or more techniques of the present disclosure. In the example of FIG. 1, system 10 may represent a computing device or computing system, such as a mobile computing device (e.g., a smartphone, a tablet computer, a personal digital assistant, and the like), a desktop computing device, a server system, a distributed computing system (e.g., a “cloud” computing system), or any other device capable of receiving patient data 18 and performing the techniques described herein.

As further described herein, mortality prediction system 10 implements a lightweight random-forest model, generally referred to herein as “mini Serious Illness Algorithm” or “minSIA,” to enable a risk-level scoring system to predict in-hospital mortality. In particular, mortality prediction engine 26 of computing system 10 applies a prediction model 15 (e.g., a random-forest “minSIA” model described herein), primarily using data available soon after admission (e.g., within 48 hours), to predict mortality of patient 8 during a specified time period after hospital admittance, for example, during six months of care in one example model described herein. In general, data input 12 receives data indicative of a plurality of factors and criteria for patient 8. Data input 12 may, for example, query patient records 24, remote databases or systems, or other sources to automatically obtain the data. Additionally or alternatively, data input 12 may receive data manually from one or more clinicians.

Based on the determined risk level, mortality prediction system 10 outputs (e.g., via display 11) report 20 to aid caregivers in the early hours of care. Report 20 may, for example, include a suggested treatment plan selected or otherwise determined by report generator 14 based on the computed risk levels.

As described in further detail below, in one example, mortality prediction engine 26 executes a machine-learning algorithm that applies a prediction model 15 to predict mortality of patient 8 over the six months after hospital admittance using only data collected directly from the patient and from diagnostics performed and available at the time of admission of the patient (e.g., within 48 hours). In one specific example, mortality prediction engine 26 applies prediction model 15 to predict, at the time of admission, the mortality risk of patient 8 over a period of six months, in some cases prior to any other diagnosis of the patient apart from the presence of a metastatic disease and whether the patient has one or more active tumors. As such, prediction model 15 is able to predict mortality of patient 8 over six months after admittance without requiring subjective estimates from clinical judgment during or after admission, without using and evaluating diagnostic imaging of the patient, and without any specialized, disease-specific testing during or after admission of the patient, since data for such imaging or disease-specific testing and evaluation may not be available for considerable time (weeks or months in some cases) after admission.

In accordance with the techniques of this disclosure, mortality prediction engine 26 receives a set of patient data parameters for patient 8 upon admission of the patient, such as up to 48 hours of hospitalization. Computing system 10 executes mortality prediction engine 26 to apply a machine-learning model (e.g., prediction model 15) that has been trained to predict patient mortality for an extended time period after admission of the patient. That is, machine-learning prediction model 15 is trained using training data that configures mortality prediction engine 26 to predict the mortality of patient 8 using only a small set of data parameters. For example, the set of data parameters may be limited to parameters that meet certain criteria or fall into certain categories. For example, the parameters may consist of only: (1) data parameters available at admission of the patient, (2) a data parameter indicative of the presence of a metastatic disease in the patient, and (3) a data parameter indicative of the presence of at least one active tumor in the patient.

In certain examples, the training set of data parameters for prediction model 15 utilizes a red cell distribution width (RDW) which, in general, is available at the time of admission, but not widely used. In one particular example, as described in detail below, machine-learning prediction model 15 is trained using a set of training data parameters that configure mortality prediction engine 26 to accurately predict the mortality of patient 8 using only eight data parameters: (1) a red cell distribution width (RDW) for the patient, (2) the age of the patient at admission, (3) a data parameter indicative of presence of a metastatic disease in the patient (METS), (4) a data parameter indicative of presence of an active tumor in the patient (METS), (5) albumin level, (6) Creatinine level, (7) platelet count, and (8) total Bilirubin.

In another example, machine-learning prediction model 15 is trained using a set of training data parameters that configure mortality prediction engine 26 to accurately predict the mortality of patient 8 when the set of data parameters available at admission of the patient applied by the machine-learning model includes data parameters from both a Complete Metabolic Panel (CMP) and a Complete Blood Count (CBC) without any other parameters other than the data parameter indicative of the presence of a metastatic disease in the patient and the data parameter indicative of the presence of at least one active tumor in the patient.

As one example, a clinical-data warehouse was used to create an electronic-medical-record-derived dataset of hospital admissions for 158,323 patients within a six-hospital network in the Twin Cities area of Minnesota. The encounters spanned a six-year period ranging from 2012-2018. The hospital system consists of one 450-bed university tertiary-care center and five community hospitals ranging from 100-450 beds in capacity. Patients who were less than 18 years of age, who did not consent to their medical record being used for research purposes, or who had less than one year of follow-up mortality data were excluded. Hospitalizations to all units and services were included as long as they met the above criteria. Vital status and death dates were obtained from the state death registry. The database included the complete death record issued from 2011 onwards for deceased individuals who were born in Minnesota, who died in Minnesota, or who ever had a permanent address in the state.

The dataset included four broad classes of variables (e.g., features) that were commonly available in the electronic medical records (EMRs) from most hospitalizations and were clinically relevant: (1) demographic variables such as age, sex, and race; (2) physiologic variables such as systolic blood pressure (SBP), diastolic blood pressure, pulse, respiratory rate, temperature, pulse-oximetry readings and body mass index; (3) biochemical variables such as serum sodium, potassium, chloride, bicarbonate, creatinine, urea nitrogen, ALT, AST, alkaline phosphatase, total bilirubin, albumin, white blood cell count, hematocrit, hemoglobin, platelet count, mean corpuscular volume, red cell distribution width, troponin, pro-BNP, INR, aPTT, and arterial blood gas results; and (4) clinical co-morbidity variables from a co-morbidity profile created for each patient across the 30 classes of diseases in the AHRQ comorbidity category index from ICD codes billed during an encounter.

All laboratory and physiologic data was obtained and time-stamped within 48 hours of hospital admission. For each data element, the first available measurement within 48 hours of hospital admission was used in the model. The primary outcome of interest was predicting whether death occurred within six months of hospital admission.

Two imputation strategies were tested to deal with missing data. The first was the k-nearest-neighbor (kNN) approach, which replaced missing data in an encounter with the values of its nearest neighbor based on a distance measure. The second was the median imputation approach, where missing values for a variable were replaced with median values for the variable. The two approaches did not significantly change the model's performance. Due to its simplicity and relatively fast computation time, the median imputation was used. The dataset was partitioned into a derivation dataset and a validation dataset with encounters selected randomly at a ratio of 0.6/0.4.

The performance of logistic regression (LR) was compared to a class of machine-learning (ML) models known as random-forest (RF) models. Due to their higher discriminative performance, the RF models were selected for further development. For example, RFs are known for their superior “out of box” performance, are able to handle non-linear data, and are less prone to over-fitting. RFs are based on decision trees. Decision tree algorithms formulate decision rules to fit the underlying data. However, decision trees are frequently “unstable,” and are sensitive to minor alterations in the data. RFs aggregate the results of many different decision trees in order to eliminate this instability. RFs typically utilize two basic strategies to achieve this objective: (1) the algorithm utilizes a random subset of the training data to build each new tree in the ensemble; and (2) a random subset of features is utilized for constructing each decision rule in a tree. This approach avoids introducing an inordinate degree of bias into the classification, stemming from a few influential observations. Variable importance is interpreted in RFs by using an importance measure known as the “Mean Decrease in Gini index” (MDGini). MDGini measures a variable's performance by randomly permuting the variable and measuring the resultant change in classification error. For each RF classifier, 501 trees were used in the ensemble of the analysis. The “mtry” parameter, which is the number of variables randomly sampled as candidates at each split, was equal to the square root of “p,” where “p” represents the number of variables in the model. The RF implementation from the “ranger” package in “R” was used for data analysis.

For non-normal variables, median values with interquartile range (IQR) were reported. Mean with standard deviation (SD) were reported for normal variables. The significance of comparisons between two non-normal continuous variables was tested using the Wilcoxon test. For comparisons between two categorical variables the Fisher Test was used.

The discriminative performance of the models was measured by constructing Receiver Operating Characteristic (ROC) curves and calculating the Area Under the Curve (AUC) on the validation dataset. In clinical studies, the AUC gives the probability that a randomly selected patient who experienced an event (e.g., a disease or condition) had a higher mortality risk score than a patient who had not experienced the event. This probability is equal to the Area Under the Receiver Operating Characteristic (AUROC) curve and ranges from 0.5 to 1. The 95% confidence intervals around the AUC estimates was estimated using the DeLong method, which is implemented in the pROC package in R. In order to evaluate whether the predicted probability of six-month mortality from the random-forest model reflected the observed probabilities, model calibration curves were constructed. In a perfectly calibrated model, all the data points would fall along a diagonal straight line.

The demographic, physiological, and laboratory characteristics of the encounters are shown in Table 1. As shown in Table 1, in 8.1% of the hospitalizations, death occurred within six months of hospital admission. The median age, creatinine, blood urea nitrogen (BUN), mean corpuscular volume (MCV), white blood count (WBC), and red cell distribution width (RDW) were higher in hospitalizations that were followed by death within six months. The albumin and hemoglobin readings were significantly lower for patients who died within six months of hospital admission.

TABLE 1

| | Survival greater than | | Survival less than | | p |
|---|---|---|---|---|---|
| | 6 months | | 6-months | | Value |
| Number of | 145478 | | 12845 | | |
| patients | | | | | |
| | 0 | (0.0) | 12845 | (100.0) | <0.001 |
| Age (years, | 50.16 | (20.02) | 72.32 | (15.79) | <0.001 |
| median [IQR]) | | | | | |
| Sex (=Female, | 92871 | (63.8) | 6423 | (50.0) | <0.001 |
| number %) | | | | | |
| Race (=White, | 119615 | (82.2) | 11299 | (88.0) | <0.001 |
| number %) | | | | | |
| Metastatic | 4530 | (3.1) | 3479 | (27.1) | <0.001 |
| Disease | | | | | |
| (number, %) | | | | | |
| Tumor | 13283 | (9.1) | 4722 | (36.8) | <0.001 |
| (number, %) | | | | | |
| Serum Albumin | 3.80 | [3.30, 4.20] | 3.10 | [2.60, 3.60] | <0.001 |
| (g/dL, median | | | | | |
| [IQR]) | | | | | |
| Total Bilirubin | 0.60 | [0.40, 0.90] | 0.70 | [0.50, 1.20] | <0.001 |
| (mg/dL, median | | | | | |
| [IQR]) | | | | | |
| Blood Urea | 15.00 | [11.00, 20.00] | 24.00 | [16.00, 37.00] | <0.001 |
| Nitrogen | | | | | |
| (mg/dL, median | | | | | |
| [IQR]) | | | | | |
| Serum Chloride | 104.00 | [101.00, 106.00] | 102.00 | [97.00, 105.00] | <0.001 |
| (mEq/l, median | | | | | |
| [IQR]) | | | | | |
| Serum | 26.00 | [24.00, 28.00] | 26.00 | [23.00, 29.00] | 0.939 |
| Bicarbonate | | | | | |
| (mEq/l, median | | | | | |
| [IQR]) | | | | | |
| Serum | 0.84 | [0.70, 1.04] | 1.05 | [0.77, 1.59] | <0.001 |
| Creatinine | | | | | |
| (mg/dl, median | | | | | |
| [IQR]) | | | | | |
| Blood Glucose | 109.00 | [94.00, 133.00] | 118.00 | [99.00, 150.00] | <0.001 |
| (mg/dl, median | | | | | |
| [IQR]) | | | | | |
| Hemoglobin | 12.70 | [11.20, 14.10] | 11.40 | [9.70, 13.10] | <0.001 |
| (mmol/l, | | | | | |
| median [IQR]) | | | | | |
| International | 1.06 | [0.99, 1.20] | 1.25 | [1.09, 1.77] | <0.001 |
| Normalized | | | | | |
| Ratio (IU, | | | | | |
| median [IQR]) | | | | | |
| Serum Lactate | 1.50 | [1.00, 2.20] | 2.00 | [1.30, 3.50] | <0.001 |
| (mmol/L, | | | | | |
| median [IQR]) | | | | | |
| Mean | 90.00 | [86.00, 93.00] | 92.00 | [87.00, 97.00] | <0.001 |
| Corpuscular | | | | | |
| Volume | | | | | |
| (femtoliters/cell, | | | | | |
| median [IQR]) | | | | | |
| Serum Sodium | 139.00 | [136.00, 141.00] | 137.00 | [134.00, 140.00] | <0.001 |
| (mEq/l, median | | | | | |
| [IQR]) | | | | | |
| Platelet count | 218.00 | [175.00, 267.00] | 203.00 | [138.00, 275.00] | <0.001 |
| (×10\hat{ }9/L, | | | | | |
| median [IQR]) | | | | | |
| pro-Brain | 1267.50 | [300.00, 4120.00] | 4130.00 | [1267.50, 11200.00] | <0.001 |
| Natriuretic | | | | | |
| Peptide (pg/mL, | | | | | |
| median [IQR]) | | | | | |
| Red Cell | 13.50 | [12.90, 14.40] | 15.20 | [13.90, 17.10] | <0.001 |
| Distribution | | | | | |
| Width (μm, | | | | | |
| median [IQR]) | | | | | |
| White Blood | 8.90 | [6.70, 12.00] | 9.60 | [6.80, 13.70] | <0.001 |
| Cell count | | | | | |
| (×10\hat{ }9/L, | | | | | |
| median [IQR]) | | | | | |
| Body Mass | 28.13 | [24.37, 32.77] | 25.10 | [21.59, 29.66] | <0.001 |
| Index (kg/m2) | | | | | |

Cohort characteristics. The table is stratified by whether death occurred within six months of hospital admission. Interquartile ranges are listed in parentheses. Median values are reported for non-normal variables. The Wilcox test is used for comparisons between the non-normal continuous variables, and the Fisher test is used for comparisons between categorical variables.
IQR: Interquartile range.
SD: Standard deviation.

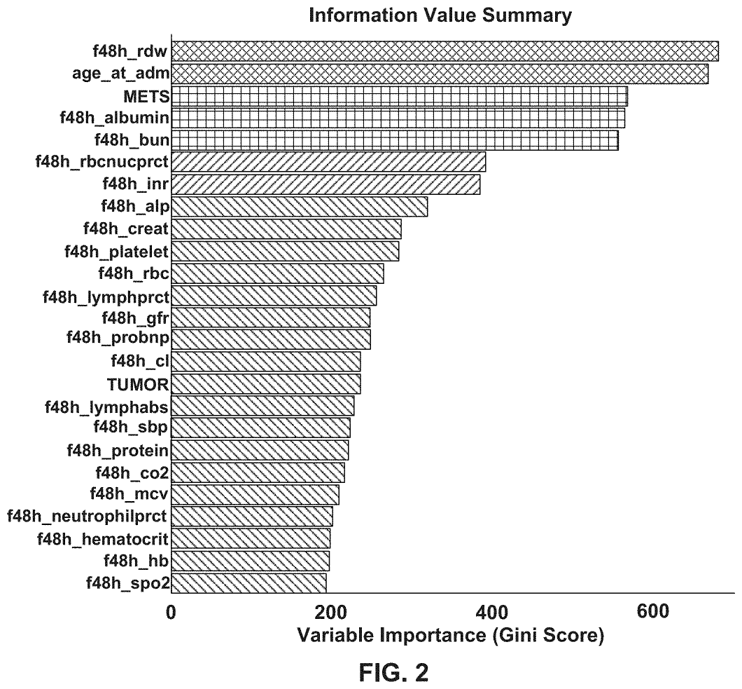

The highest-ranking 25 features of one example algorithm, referred herein to as the “Serious Illness Algorithm” (SIA), are shown in FIG. 2. In this example, red cell distribution width, age, presence of metastatic disease, serum albumin, and BUN were the highest-ranking variables. In FIG. 2, the 25 highest-ranking features in the SIA model are ranked by importance as measured by the “Mean Decrease in Gini” index. The “f48_” prefix refers to values obtained within first 48 hours of hospitalization of a patient.

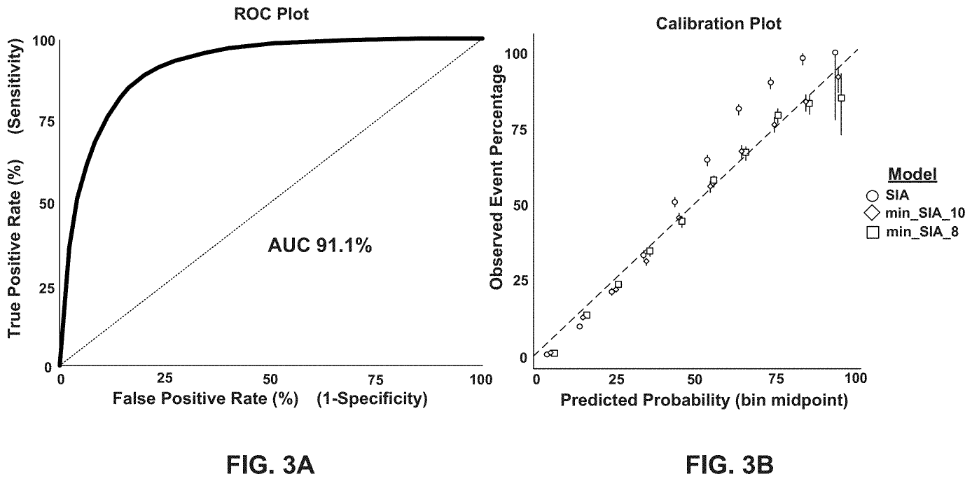

The SIA model with all available predictors in the dataset (54 predictors) had an AUC of about 0.94 (0.93-0.95). The leaner (e.g., more lightweight) models with 8 and 10 variables (“minSIA-8” and “minSIA-10,” respectively), had AUCs of about 0.92 (0.91-0.92) and 0.93 (0.91-0.93), respectively. The ROC curve for minSIA-8 is shown in FIG. 3A. FIG. 3B is a graph illustrating the observed rate of patient death at one year from hospitalization (y-axis) for each of the 10 probability bins. The predicted probability from the random-forest model is indicated on the x-axis. The dotted diagonal line represents theoretical points along a perfectly calibrated model. Each point on the graph represents one of the 10 bins of probability. The bars delineate the 95% confidence intervals around the observed probability.

The calibration of a model is a measure of how well the probabilities estimated by the model reflect the observed probabilities. minSIA-8 and minSIA-10 had excellent calibration across the whole probability range. Even the though the SIA had a higher AUC value, the minSIA models were better calibrated.

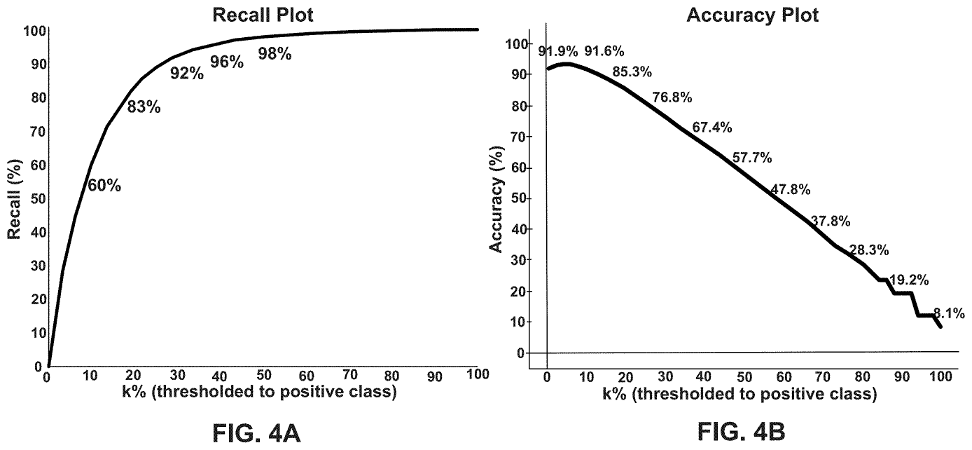

FIG. 4A is a graph illustrating an example “recall” plot, which shows the percentage of the overall number of cases in a given category “gained” (y-axis) when the minSIA-8 is applied and the highest k-deciles (x-axis) are selected. For example, if the positivity threshold is set to be the highest ranking 20% cases (by predicted probability) then 83% percent of true positives would be selected. FIG. 4B is a graph illustrating the “accuracy” plot, which plots the accuracy (y-axis) of the model at each decile threshold of predicted probability (x-axis).

The cumulative gains or recall plot (left panel of FIG. 4A) visualizes the percentage of targets selected at a certain threshold of predicted probability (k%). For example, if patients within the top 20% of predicted probability range were selected, then 83% of the patients that died within six months would be “captured” with the minSIA-8 model (left panel of FIG. 4A). For the full SIA model, this number would be 88% (not shown in FIG. 4A or FIG. 4B). At a threshold of the top 20% of predicted probability (k=20%), the accuracy of the minSIA-8 is about 85.3% (right panel of FIG. 4B).

The model development and testing may be repeated using either of two approaches. In a first approach, each distinct hospitalization may be treated as a unit of analysis. In the first approach, the last set of data from each available hospitalization for each patient may be used. In a second approach, each unique patient may be treated as a unit of analysis. In the second approach, the dataset may be sampled and one hospitalization for each patient may be randomly selected for inclusion analysis (i.e. random admission model). This may be done, for example, to test the effect of potential selection bias that could be theoretically introduced by using multiple data points from the same patient. Both these strategies yield models with nearly identical AUCs and predictive performance.

According to the techniques of this disclosure, it is possible to accurately identify patients who have a higher risk of six-month mortality at the time of hospital admission by constructing and validating minSIA-8, which is a high-performing and “lightweight” model. “Lightweight” as used herein refers to the relatively small number of data inputs utilized by the model to produce highly accurate results. For example, minSIA uses data that is typically available to clinicians during the first 48 hours of hospital admission, and delivers remarkable predictive performance. In fact, SIA and minSIA may have among the highest-known AUCs described for predictive models in multi-condition, hospitalized patients. The minSIA relies on 8 predictive factors and is relatively easy to use for clinicians. The probability estimates produced by the model closely mirror the observed rates of mortality as demonstrated in the calibration curve.

In many cases, clinicians may be relatively poor at estimating the probability of patient survival beyond a few days, even for intensive-care-unit (ICU) patients. For example, in prior studies, clinician predictive ability has ranged from an AUC of 0.5 to 0.79 for six-month survival. By comparison, the minSIA has an excellent AUC at about 0.92. Other studies of at longer-term mortality estimation (e.g., 3-12 months) in multi-condition, hospitalized patients have achieved an AUC of around 0.94 with a deep-learning approach and 0.91 with random-forest models. However, both these studies used a much larger number of predictors than minSIA and relied on additional data beyond that which was available at the time of hospital admission, thereby limiting their use at the beginning of a hospitalization.

Even though the SIA has a higher AUC (e.g., about 0.94) than the minSIA, the minSIA may be better-calibrated and uses significantly fewer variables as input. The difference in predictive performance between the two models is not statistically significant enough to be clinically meaningful. The minSIA model retains the excellent performance of the SIA, but achieves this result with fewer variables.

When these models are deployed at a system-wide level (such as with automatic EMR interfacing), it is possible to identify about 83%-88% of patients that will die within six months of hospital admission by screening patients in the top-2-decile of predicted probability (left panel of FIG. 3). The application of this model would facilitate automated flagging of high-risk patients for clinical review. Such a strategy could help enable a majority of patients that could benefit from a serious-illness consultation to be identified in a timely manner.

It is notable that red-cell-distribution width emerged as the single-most-significant variable in the prognostic model, even outperforming age as a prognostic factor. While previous studies have shown that red-cell-distribution width (RDW) is linked with mortality, initial applications of the present techniques have confirmed that RDW is central to mortality prognostication.

The example models of this disclosure were developed and validated on a demographically, economically, and clinically diverse cohort. The validation dataset includes data from a large, multi-hospital health system. The system encompasses a university tertiary-care center and urban, suburban, and semi-rural hospitals. Ultimately, the models may need to be validated in other settings in order to further demonstrate geographic and temporal portability. For validation purposes, state death-registry data may be used for ascertaining the dates of patients' deaths (e.g., for out-of-hospital deaths). If a patient's death is not reported to a pertinent state registry, then it may not be captured in the validation dataset.

It is notable that two ICD-derived variables were used in developing the minSIA model: the presence of metastatic disease and tumor(s). ICD codes are typically submitted at the end of a patient's hospitalization. However, with the exception of a relatively small number of new cancer cases diagnosed during the index hospitalization, this data would be available to clinicians at the time of admission as part of their clinical assessment.

The present disclosure demonstrates that it is possible to develop high-performance, parsimonious, predictive models, such as minSIA-8, to accurately identify patients at high risk for six-month mortality at the time of hospital admission. This could potentially be used in areas where accurate risk-stratification is crucial, such as institutional implementations of serious-illness care programs and outcomes research. Additional future work may be needed to test how to incorporate this model into the clinical workflow in order to enable timely serious-illness care conversations in appropriate situations. Care will have to be taken that any such model implementation is part of a comprehensive serious-illness care program designed around the bedrock principles of patient autonomy, beneficence, non-maleficence, justice, privacy, and confidentiality.

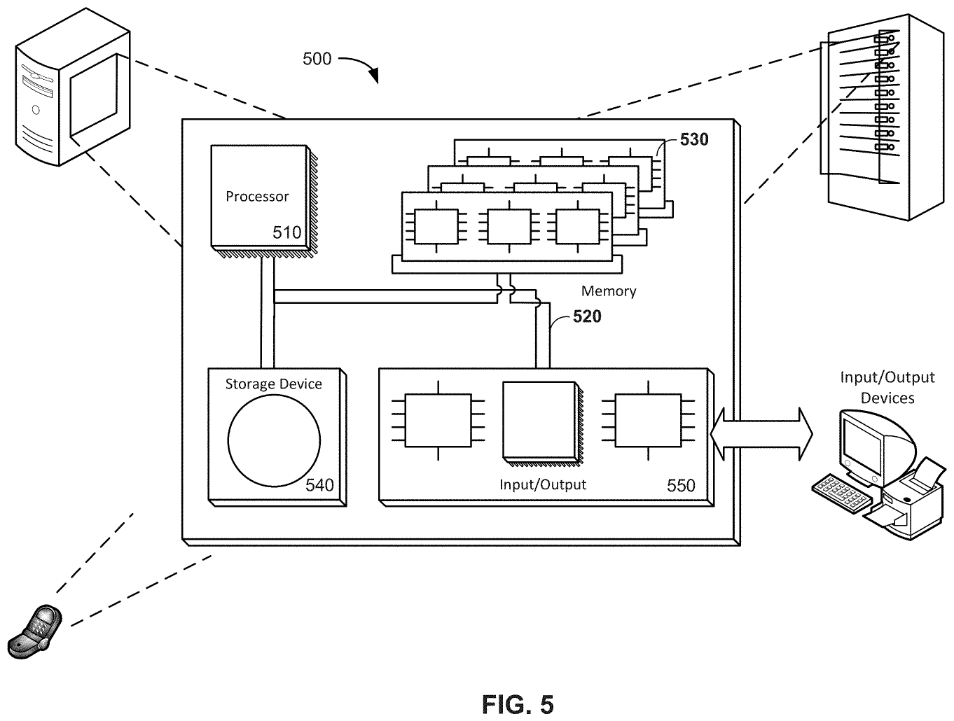

FIG. 5 is a block diagram illustrating a detailed example of various devices that may be configured to implement one or more techniques of the present disclosure. That is, device 500 of FIG. 5 provides an example implementation for the mortality prediction system 10 of FIG. 1 for predicting in-hospital mortality for patients. Device 500 may be a mobile device (e.g., a tablet, a personal digital assistant, or other mobile device), a workstation, a computing center, a cluster of servers, or other examples of a computing environment, centrally located or distributed, that is capable of executing the techniques described herein. Any or all of the devices may, for example, implement portions of the techniques described herein for generating and outputting predicted prostate cancer visualizations for display. In some examples, functionality of morality prediction system 10 may be distributed across multiple computing devices, such as a cloud-based computing system for computing the predicted scores and generating the reports, and a client device, such as a table or mobile phone, for accessing and viewing the reports.

In the example of FIG. 5, computer-implemented device 500 includes a processor 510 that is operable to execute program instructions or software, causing the computer to perform various methods or tasks, such as performing the techniques for generating and/or using multiparametric models for prostate cancer prediction as described herein. Processor 510 is coupled via bus 520 to a memory 530, which is used to store information such as program instructions and/or other data while the computer is in operation. A storage device 540, such as a hard disk drive, nonvolatile memory, or other non-transient storage device stores information such as program instructions, data files of the multidimensional data and the reduced data set, and other information. The computer also includes various input-output elements 550, including parallel or serial ports, USB, Firewire or IEEE 1394, Ethernet, and other such ports to connect the computer to external devices such a printer, video camera, display device, medical imaging device, surveillance equipment or the like. Other input-output elements include wireless communication interfaces such as Bluetooth, Wi-Fi, and cellular data networks.

The computer itself may be a traditional personal computer, a rack-mount or business computer or server, or any other type of computerized system. The computer, in a further example, may include fewer than all elements listed above, such as a thin client device or a mobile device having only some of the shown elements. In another example, the computer is distributed among multiple computer systems, such as a distributed server that has many computers working together to provide various functions.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media, which includes any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable storage medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

### ABBREVIATIONS

aPTT: activated partial thromboplastin time

AST: Aspartate Amino Transferase

ALT: Alanine Amino Transferase

AHRQ: Agency for Health Care Research and Quality

EMR: Electronic Medical Record

MCV: Mean Corpuscular Volume

WBC: White Blood Cell count

CMP: Complete Metabolic Panel

CBC: Complete Blood Count

BMP: Basic Metabolic Panel

BNP: Brain Natriuretic Peptide

RF: Random Forest

Lab: Laboratory

LR: Likelihood Ratio

Logistic regression: LR

SD: Standard Deviation

AUC: Area under the Curve

ROC: Receiver operator curve

OOB: Out of bag

ICD9-CM: International Classification of Diseases 9—Clinical Modification

ICD10: International Classification of Diseases 10

INR: international normalized ratio

MD-Gini: Mean Decrease in Gini Index

Machine Learning: ML

What is claimed is:

1. A system comprising:
a data repository configured to store one or more of a set of patient data parameters for a patient; and
a computing system executing a mortality prediction engine configured to apply a machine learning model trained to predict mortality of the patient at a time period after admission of the patient, wherein the machine learning model is trained based on training data that configures the mortality prediction engine to predict the mortality of the patient using one or more of the set of data parameters, wherein the set of data parameters consists only of data parameters that meet at least one of the following criteria:
data parameters available at admission of the patient;
data parameters indicative of a presence of a metastatic disease in the patient; or
data parameters indicative of a presence of at least one active tumor in the patient.

2. The system of claim 1, wherein the data parameters available at admission of the patient consist only of data parameters from a Complete Metabolic Panel (CMP) and data parameters from a Complete Blood Count (CBC).

3. The system of claim 1, wherein the data parameters available at admission of the patient comprise a red cell distribution width (RDW) for the patient.

4. The system of claim 1, wherein the set of data parameters consists of the following eight data parameters:
a. red cell distribution width (RDW) for the patient,
b. age of the patient at admission,
c. a data parameter indicative of a presence of a metastatic disease in the patient (METS),
d. a data parameter indicative of a presence of an active tumor in the patient (METS),
e. albumin level,
f. Creatinine level,
g. Platelet count, and
h. total Bilirubin.

5. The system of claim 1, wherein the time period comprises six months after admission of the patient.

6. The system of claim 1, wherein the computing system comprises one or more of a cloud-based computing platform, a mobile device, a laptop, or a server.

7. The system of claim 1, wherein the computing system is further configured to output a report indicative of the predicted mortality of the patient.

8. A method comprising:
receiving a set of patient data parameters for a patient upon admission of the patient;
executing, by a computing system, a mortality prediction engine to apply a machine learning model trained to predict mortality of the patient at a time period after admission of the patient, wherein the machine learning model is trained based on training data that configures the mortality prediction engine to predict the mortality of the patient using one or more of the set of data parameters, wherein the set of data parameters consists only of data parameters that meet at least one of the following criteria:
data parameters available at admission of the patient;
data parameters indicative of a presence of a metastatic disease in the patient; or
data parameters indicative of a presence of at least one active tumor in the patient; and
outputting a report indicative of the predicted mortality of the patient.

9. The method of claim 8, wherein the data parameters available at admission of the patient consist only of data parameters from a Complete Metabolic Panel (CMP) and data parameters from a Complete Blood Count (CBC).

10. The method of claim 8, wherein the data parameters available at admission of the patient comprise a red cell distribution width (RDW) for the patient.

11. The method of claim 8, wherein the set of data parameters consists of the following eight parameters:
a. red cell distribution width (RDW) for the patient,
b. age of the patient at admission,
c. a data parameter indicative of a presence of a metastatic disease in the patient (METS),
d. a data parameter indicative of a presence of an active tumor in the patient (METS),
e. albumin level,
f. Creatinine level,
g. Platelet count, and
h. total Bilirubin.

12. The method of claim 8, wherein the time period comprises six months after admission of the patient.

13. The method of claim 8, wherein the computing system comprises one or more of a cloud-based computing platform, a mobile device, a laptop, or a server.

14. A non-transitory computer-readable medium having program code for causing a processor to:
receive a set of patient data parameters for a patient upon admission of the patient;
execute a mortality prediction engine to apply a machine learning model trained to predict mortality of the patient at a time period after admission of the patient, wherein the machine learning model is trained is trained based on training data that configures the mortality prediction engine to predict the mortality of the patient using the set of data parameters, wherein the set of data parameters consists only of data parameters that meet at least one of the following criteria:
data parameters available at admission of the patient;
a data parameters indicative of a presence of a metastatic disease in the patient; or
data parameters indicative of a presence of at least one active tumor in the patient; and
output a report indicative of the predicted mortality of the patient.

15. The computer-readable medium of claim 14, wherein the data parameters available at admission of the patient consists only of data parameters from a Complete Metabolic Panel (CMP) and data parameters from a Complete Blood Count (CBC).

16. The computer-readable medium of claim 14, wherein the data parameters available at admission of the patient comprises a red cell distribution width (RDW) for the patient.

17. The computer-readable medium of claim 14, wherein the set of data parameters consists of the following eight parameters:
a. red cell distribution width (RDW) for the patient,
b. age of the patient at admission,
c. a data parameter indicative of a presence of a metastatic disease in the patient (METS),
d. a data parameter indicative of a presence of an active tumor in the patient (METS),
e. albumin level,
f. Creatinine level,
g. Platelet count, and
h. total Bilirubin.

18. The computer-readable medium of claim 14, wherein the time period comprises six months after admission of the patient.

19. The computer-readable medium of claim 14, wherein the computing system comprises one or more of a cloud-based computing platform, a mobile device, a laptop, or a server.
